(19) United States
(12) Patent Application Publication
(10) Pub. No.: US 2007/0248675 A1
(43) Pub. Date: Oct. 25, 2007

(54) Composite comprising polysaccharide-functionalized nanoparticle and hydrogel matrix, a drug delivery system and a bone defect replacement matrix for sustained release comprising the same, and the preparation method thereof

(75) Inventors: Gi yoong Tae, Gwangju (KR); Yong-Il Chung, Gwangju (KR); Jong-Ho Lee, Seoul (KR); Yong Doo Park, Seoul (KR)

Correspondence Address: ROTHWELL, FIGG, ERNST & MANBECK, P.C., 1425 K STREET, N.W., SUITE 800, WASHINGTON, DC, 20005, US

(73) Assignee: Gwangju Institute of Science and Technology, Gwangju (KR)

(21) Appl. No.: 11/783,339

(22) Filed: Apr. 9, 2007

Related U.S. Application Data
(63) Continuation-in-part of application No. 11/391,480, filed on Mar. 29, 2006.

(30) Foreign Application Priority Data
Sep. 8, 2005 (KR) 10-2005-0083763
Aug. 21, 2006 (KR) 10-2006-0078894

(51) Int. Cl. A61K 9/00; A61K 38/16

(52) U.S. Cl. 424/486; 514/12

(57) ABSTRACT

The present invention relates to a nanoparticle-protein-hydrogel composite comprising (1) a polysaccharide-functionalized nanoparticle comprising a core composed of a biodegradable polymer, a hydrogel surface layer composed of a biocompatible polymer emulsifier, and a polysaccharide physically bound to the core and/or the hydrogel layer; (2) a protein forming a specific binding with the polysaccharide; and (3) a hydrogel matrix composed of a biocompatible polymer as a matrix for the nanoparticle. The present also relates to a drug delivery system and a bone defect replacement matrix comprising the composite for sustained release, and the preparation method thereof. Further, the present invention also provides a method for controlling the release rate of a protein drug by changing the content of the polysaccharide in a unit mass of the nanoparticle and/or by changing the content of the nanoparticle in a unit mass of the composite.

### CROSS REFERENCE TO RELATED APPLICATIONS

This is a continuation-in-part of co-pending U.S. patent application Ser. No. 11/391,480, filed on Mar. 29, 2006 entitled “Polysaccharide-functionalized nanoparticle, drug delivery system for controlled release comprising the same and preparation method thereof”, which claims priority under 35 U.S.C. § 119 based on Korean patent application no. 10-2005-0083763 filed Sep. 8, 2005, all of which are incorporated herein by reference in its entirety. This application also claims priority under 35 U.S.C. § 119 based on Korean patent application no. 10-2006-0078894 filed Aug. 21, 2006, which is incorporated herein in its entirety.

### TECHNICAL FIELD

The present invention relates to a nanoparticle-protein-hydrogel composite, a drug delivery system and a bone defect replacement matrix comprising the composite for sustained release, and the preparation method thereof. The present invention also provides a method for controlling the release rate of a protein drug by changing the content of the polysaccharide in a unit mass of the nanoparticle and/or by changing the content of the nanoparticle in a unit mass of the composite.

### RELATED PRIOR ART

A bone defect replacement matrix plays a substitutional role for regenerating bone lost through disease or injury, and preferably aims to help new bone tissue grow and replace damaged parts instead of aiming just to fill the defect sites. The bone defect replacement matrix may be used for osteogenic promotion and substitution for injured bone tissues, most notably found in the cases of limb or spine fractures, fracture dislocations, non-union, delayed union, osteomyelitis, and tumor ablation in orthopedics, as well as alveolar defects in dentistry.

Currently, bone grafting materials are widely implanted into the defect sites, and autogenous bone, allogeneic bone, xenogeneic bone and synthetic bone are used as the bone grafting materials.

First, the autogenous bone graft is performed by using autogenous bone separated from the patient and cultivated. The autogenous bone graft has advantages of (i) superior osteoinductive activity and (ii) recovery of implanted bone and rapid conversion into viable bone, and (iii) separability into various shapes depending on the use [J. Foot Ankle Surg. 1996; 35:413-7], while showing drawbacks of (i) quantity limitation, (ii) additional operation at donating region and prolonged operation time, and (iii) bone defect, nerve damage, possibility of disease and prolonged recovery period at donating region [Clin. Orthop. 1996; 329:300-9, Spine 1995; 20:1055-60, J. Bone Joint Surg. Br. 1988; 70:431-4, Br. J. Neurosurg 2000; 14:476-9, J. South Orthop. Assoc. 2000; 9:91-7, Spine 2000; 25:2400-2, J. Bone Joint Surg. Br. 1989; 71-B:677-80, J. Orthop. Trauma 1989; 3:192-5].

The allogeneic or xenogeneic bone graft does not necessitate the second operation, and has advantages of (i) shortened operation time and recovery period, along with (ii) low-price material compared to synthetic bone grafting material [AORN J. 1999; 70:660-70, Orthop. Clin. North Am. 1999; 30:685-98]. However, it has drawbacks of (i) about twice prolonged osteoinductive period compared to autogenous bone, (ii) a large amount of resorption during ossification process, (iii) the inferior quality of regenerated bone and (iv) possibility of immune reaction or infection [Clin. Orthop. 1972; 18:19-27, J. Bone Joint Surg. Am. 1983; 65-A:239-46, J. Appl. Biomater. 1991; 2:187-208, J. Arthroplasty 2000; 15:368-71, J. Bone Joint Surg. Br. 2001; 83(1):3-8, J. Bone Joint Surg. Br. 1999; 81:333-5, Orthop. Clin. North Am. 1987; 18:235-9].

Besides, the synthetic bone graft is performed by using material such as hydroxyapatite, tricalcium phosphate, calcium aluminate, plastics and metals, and has an advantage of low antigen-antibody reaction. However, it has drawbacks of inferior bone formation into a matrix and cytotoxicity and non-biocompatibility. The widely accepted combination use of the synthetic bone with autogeneous bone also have limitation of high resorption, low bone regeneration, and each particle may be encompassed by fibrous tissue, thus failing to show clinically satisfactory effect [Biomaterials 2000; 21:2615-21, Clin. Orthop. 1989; 240:53-62, Orthop. Clin. North Am. 1999; 30:591-8]. Further, the aforementioned bone implantation methods have a deformation problem after operation in common.

Recently, there has been an attempt made to help the bone defect regenerate by using a molecular therapy. This therapy is an approach of inducing activation of initial step for accelerating the tissue regeneration by supplying functional protein molecules such as a growth factor, signaling molecules, a transcription factors and other effectors to damaged tissue region for a predetermined period of time. Especially, the tissue regeneration through the delivery of a growth factor is very important from a tissue-engineering viewpoint. BMP is widely used as a target growth factor with regard to bon generation. The method using BMP is expected eventually to be superior in bone regeneration to the autogenous bone grafting method.

BMP was first described by Urist as a bone matrix protein involved in bone formation [Science 1965; 150: 893-899]. Over fifteen BMP family members have currently been identified [Science 1988; 242: 1528-1534], and belong to the TGF-β (transforming growth factor beta) superfamily. In addition to bone formation, BMP is diversely involved in cell division, apoptosis, cell migration, differentiation [Genes Dev. 1996; 10(13): 1580-94], the development of limb buds during embryogenesis [Mech Dev 1997; 69(1-2): 197-202], ectopic bone formation [Acta Orthop Scand 1996; 67(6): 606-10], and differentiation of mesenchymal progenitor cells to osteoblasts or chondrocytes [J. Cell Biochem 1997; 66(3): 394-403, J. Cell Biol 1998; 140(2): 409-18, Bone 1998; 23(3): 223-31, Exp Cell Res 1999; 251(2): 264-74].

Together with selection of a target growth factor, the effective delivery and release of target proteins is especially important in tissue regeneration using growth factors. In general, during the natural healing process of an injured tissue, proliferation and increases in the synthesis of extracellular matrix molecules occur at the edge of the lesion, but only temporarily and within a limited manner, due to the lack of a sustained supply of signaling molecules [J. Orthop Sports Phys Ther 1998; 28(4): 192-202]. To overcome limitations of the natural healing process it is necessary to develop a sustained delivery system, one that can continuously supply active signaling molecules.

Although there have been attempts made to develop various sustained release system for local delivery of a growth factor for the last several years, any ideal system has not been developed until now. A matrix-based drug delivery system was recently developed and is being applied to the regeneration of various tissues other than bone tissue. The matrix-based system needs to be basically non-immunogenic, non-toxic, biocompatible, biodegradable and easily manufactured. Further, the matrix-based system needs to stabilize the loaded signaling molecules, control their release, and also support the structural strength as a template filling the lesion site. For bone regeneration, various materials used in conjunction with BMP delivery have been applied as a potential matrix.

Hydroxyapatite (‘HAP’ hereinafter, Ca₁₀(PO₄)₆(OH)₂), a main constituent ingredient of bone, is most commonly used among inorganic materials [Spine 1999; 15: 1179-1185, J. Biomed. Mater. Res. 2000; 51: 491-499]. Besides, β-tricalcium phosphate (β-TCP, β-Ca₃(PO₄)₂), calcium phosphate-based cement (CPC), calcium sulfate, metal and bioglass are also included in the inorganic material [In Society for Biomaterials, 6th World Biomaterials Congress 2000: p. 1135, J. Orthop Res. 2003; 21(6): 997-1004, J. Biomed. Mat. Res. 1997; 35: 421-432, U.S. Pat. Nos. 4,596,574, 4,619,655].

Although a matrix constructed only with HAP has advantages of superior cell attachment of osteoblast and calcification of tissue, the tight binding between HAP and BMP can result in the lack of bone induction. Bone defect sites may not be filled completely, and the fragility of matrix is also a problem. To overcome these problems, it was also attempted to further incorporate β-TCP or collagen in a matrix, thus being capable of controlling the rate of matrix resorption, and the use of porous matrix also improved the bone induction [Spine 1999; 15: 1179-1185, Clin. Orthop. 1988; 234: 250-254, Int. Orthop. (SICOT) 1996; 20: 321-325, J. Med. Dent. Sci. 1997; 44: 63-70, U.S. Pat. Nos. 5,001,169, 5,352,715].

CPC improved the drawbacks of the conventional systems in that it may be formulated into an injection and may fill bone defect sites. However, heat generated during the hardening process may inactivate BMP, and the effect may be reduced. Further, the radiation impermeability of CPC makes the radiological analysis difficult [J. Oral Maxillofac. Surg. 1999; 57: 1122-1126, Biomaterials 2003; 24: 2995-3003].

Natural polymeric materials such as collagen, fibrin, alginate and hyaruronic acid have also been applied.

Collagen is one of the widely clinically used materials because it may be formulated into a sponge-like shape and the development of technique for removing teimmunogenic telopeptide has made possible the minimization of foreign body reaction against raw material of collagen although the raw material is obtained from foreign species [J. Bone Jt Surg Am 2002; 84-A:2123-34, Spine 2003; 28:372-7, J. Bone Jt Surg Br 1999; 81:710-8, Spine 2002; 27:2654-61, EP 0206801, U.S. Pat. Nos. 4,394,370, 4,975,527]. However, collagen-based systems necessitate the excess Ioding of expensive BMP due to a large initial burst of the loaded growth factor, thus causing the financial burden [Trends Biotechnol. 2001; 19(7):255-265]. Drastic change in the initial concentration of a growth factor may also generate potential danger of disease transition [Clin Orthop Relat Res 1990; 260:263-79, Nat Med 1998; 4:141-4, Nature 1998; 391:320-4].

Fibrin is polymeric adhesive called fibrin glue in clinical use. Fibrin is formed during blood coagulation, and plays an important role in hemostasis and wound healing. A modified fibrin gel containing heparin was developed to achieve the sustained release of growth factors based on heparin-binding affinity, where artificial peptides with high heparin-binding affinity were covalently bound to a fibrin gel [J. Control Release 2000; 65(3): 389-402, U.S. Pat. Nos. 6,468,731, 6,723,344]. Although fibrin hydrogel per se is not always a system for sustained release, it may exceptionally function as a sustained release system when a target growth factor is nonglycosylated BMP-2 due to low solubility of protein in hydrogel [J. Orthop Res 22 (2004) 376-381].

Alginate belonging to polysaccharide is an anionic natural polymeric material, which is a copolymer of L-glucuronic acid and D-mannuronic acid. Although alginate may easily form hydrogel through the binding with Ca⁺⁺ ion, the biological activity of cell, protein and DNA may be seriously damaged during the formation of hydrogel. Further, macromolecules may easily diffuse due to the relatively large size of pores in hydrogel [Adv Drug Deliv Rev 1998; 31(3): 267-85, U.S. Pat. No. 6,748,954].

Hyaruronic acid is a polysaccharide having characteristic physicochemical and biological property. Hyaruronic acid specifically recognizes many proteins in an extracellular matrix, and stabilizes an extracellular matrix through an interaction with proteoglycan [J. Intern Med 1997; 242(1): 27-33]. Hyaruronic acid may interact with the cell surface that affects the cell behavior, and is also involved in the change of cell mobility [FEBS Lett 1998; 440(3): 444-9]. A hyaruronic acid based matrix for local delivery of BMP was reported to be prepared by using a chemical cross-linkage or by introducing hydrophobic functional group [J. Control Release 1999; 61(3): 267-79, J. Biomed Mater Res 1999; 47(2): 152-69, J. Biomed Mater Res. 2002; 59(3): 573-84, WO 0128602]. Although the latter enables the construction of a system for sustained release, the stereostructure of protein may be instabilized in the interface within a matrix.

Besides the inorganic material or the natural polymeric material, various synthetic polymers have been employed to develop a bone-formation matrix with BMP delivery. Polyesters such as PLGA(poly(DL-lactide-co-glycolide)), PLA(poly(L-lactide)) and PGA(polyglycolide) are most widely used [J. Vet Med Sci 1998; 60(4):451-8, Bone 2003; 32(4):381-6, J. Bone Joint Surg Am 1999; 81(12):1717-29, J. Biomed Mater Res 1999; 46(1):51-9, J. Biomed Mater Res 2002; 61(1):61-5, J. Biomed Mater Res 2000; 50(2):191-8, J. Biomed Mater Res 1999; 45(1):36-41, U.S. Pat. Nos. 4,186,448, 4,563,489, 5,133,755]. Polyanhydride, polyphosphazenes, polypropylene fumarate, polyethylene glycol-PLA, poloxamer and polyphosphate polymer are also utilized [J. Biomed Mater Res 1990; 24:901-11, Adv Drug Deliv Rev 2003; 55(4):467-82, Clin Orthop 1999; 41(367 suppl.):S118-29, Clin Orthop 1993; 109(294):333-43, Plast Reconstr Surg 2000; 105(2):628-37, J. Biomed Mater Res 1997; 34:95-104, U.S. Pat. No. 4,526,909].

The synthetic polymers may be degraded by the function of enzymes or cells, and may also be easily processed, thus enabling to control the porosity and the shape of a matrix. On the other side, the acidification due to the polymer degradation may cause cytotoxicity on the surrounding tissues, resulting in severe acute inflammation or chronic inflammation in the case of polymer with high molecular weight. If the in vivo degradation pattern of a polymer is bulk erosion, it is difficult to provide a sustained release system [Biomaterials 2000; 21:1837-1845, Macromolecules 1987; 20:2398-403, J. Control Release 1991; 16:15-26], and proteins may undergo structural denaturation when BMP is entrapped in a matrix.

### BRIEF DESCRIPTION OF DRAWINGS

FIG. 1 shows cumulative releases (%) of bone morphogenetic protein (‘BMP’ hereinafter) from the sustained-release polysaccharide-functionalized nanoparticles for delivering BMP according to an embodiment of the present invention.

FIG. 2 shows cumulative releases (%) of BMP from the sustained-released functional nanoparticle-hydrogel composite for delivering BMP according to an embodiment of the present invention.

FIG. 3 shows calvarial bone defects in a rat and a series of process for implanting into the defects a bone defect replacement matrix for sustained release according to an embodiment of the present invention.

FIG. 4 is the radiological evaluation of osteogenetic activity by using a soft x-ray (A: Comparative Example, B: an embodiment of the present invention).

FIG. 5 is the histological analysis of osteogenetic activity (A: Comparative Example, B: an embodiment of the present invention).

### DETAILED DESCRIPTION OF INVENTION

To overcome the aforementioned problems, the present invention aims to provide a composite comprising polysaccharide-functionalized nanoparticle and hydrogel matrix along with a drug delivery system and a bone defect replacement matrix for sustained release comprising the same, and the preparation method thereof. The drug delivery system and the bone defect replacement matrix herein show a remarkably improved sustained-release property without presenting an initial burst.

Furthermore, the present invention also aims to provide a method of controlling the release rate of a protein drug by changing the content of the polysaccharide in a unit mass of the nanoparticle; and/or by changing the content of the nanoparticle in a unit mass of the composite.

According to one aspect of the present invention, there is provided a nanoparticle-protein-hydrogel composite comprising: (a) a polysaccharide-functionalized nanoparticle comprising: (1) a core composed of a biodegradable polymer, (2) a hydrogel surface layer composed of a biocompatible polymer emulsifier, and (3) a polysaccharide physically bound to the core and/or the hydrogel layer; (b) a protein forming a specific binding with the polysaccharide; and (c) a hydrogel matrix composed of a biocompatible polymer as a matrix for the nanoparticle.

According to another aspect of the present invention, there is provided a drug delivery system for sustained release, which comprises a composite according to the present invention and an effective amount of at least one protein drug selected from the group consisting of a growth factor, a chemokine, an extracellular matrix protein and an antithrombin III. Especially, the protein drug may be a growth factor related to the bone formation, which is selected among BMP, a transforming growth factor-beta (‘TGF-beta’ hereinafter), a vascular endothelial growth factor (‘VEGF’ hereinafter), a fibroblast growth factor (‘FGF’ hereinafter) and a platelet-derived growth factor (‘PDGF’ hereinafter).

According to still another aspect of the present invention, there is provided a bone defect replacement matrix for sustained release, which comprises a sustained release system of a growth factor according to the present invention.

In a preferable embodiment, a sustained release system of a growth factor herein comprises: (a) a polysaccharide-functionalized nanoparticle comprising: (1) a core composed of a biodegradable polymer selected from the group consisting of the group consisting of poly(D,L-lactide-co-glycolide) (‘PLGA’ hereinafter), poly(lactic acid), poly(glycolic acid), poly(ε-caprolactone), poly(δ-valerolactone), poly(β-hydrobutyrate), poly(β-hydroxyvalerate) and a combination thereof, (2) a hydrogel surface layer composed of a biocompatible polymer emulsifier selected from the group consisting of a poloxamer, a poloxamine, a poly(vinyl alcohol), a poly(ethylene glycol) ether of an alkyl alcohol and a combination thereof, and (3) a polysaccharide physically selected from the group consisting of a heparin, an alginate, a hyaruronic acid, a chitosan and a combination thereof, which is bound to the core and/or the hydrogel layer; (b) an effective amount of a growth factor selected from the group consisting of BMP, TGF-beta, VEGF, FGF, PDGF and a combination thereof, which forms a specific binding with the polysaccharide; and (c) a hydrogel matrix composed of a biocompatible polymer selected from the group consisting of a poly(ethylene glycol), a poloxamer, a poly(organophosphazene), an oligo(poly(ethylene glycol)fumarate), a collagen, a gelatin, a fibrin, a hyaruronic acid, an alginate and a combination thereof as a matrix for the nanoparticle.

In another preferable embodiment, a bone defect replacement matrix for sustained release herein further comprises at least one selected from the group consisting of (i) autogenous bone without cells, allogeneic bone and xenogeneic bone; and (ii) HAP, tricalcium phosphate, calcium aluminate, 1-TCP, CPC, calcium sulfate and bioglass® for promoting the bone formation. Further, a bone defect replacement matrix for sustained release herein may further comprises (iii) a cell-binding protein for enhancing the initial affinity between the bone defect replacement matrix and bone cells and minimizing the foreign body reaction, resulting in the promoted bone formation, along with (iv) a degradable peptide linker for accelerating the degradation of hydrogel. A bone defect replacement matrix for sustained release according to the present may be used for the treatment or prophylaxis of osteoporosis, fracture of a bone, fracture dislocation, non-union, delayed union, bone defect, alveolar bone defect and a combination thereof.

As used herein, the term “a biodegradable polymer” refers to a polymer that may be degraded within an acceptable period of time in a physiological solution of pH 6-8, preferably in human body fluids.

Examples of the biodegradable polymer include a poly(lactide-co-glycolide) of Formula (1), a poly(lactic acid), a poly(glycolic acid), a poly(ε-caprolactone), poly(δ-valerolactone), poly(β-hydrobutyrate), poly(β-hydroxyvalerate) and a combination thereof. A biodegradable polymer herein is not limited to the aforementioned examples insofar as it is appropriate for preparing polysaccharide-functionalized nanoparticles after added to an aqueous emulsifier solution containing polysaccharide. Preferably, poly(lactide-co-glycolide), which has been approved by FDA as non-cytotoxic, may be used among these polymers.

As used herein, the term “a combination” of ‘oligomers and/or polymers’ refers to any kind of copolymer thereof as well as a blend thereof in melt or liquid phase. As used herein, the term “a combination” of ‘monomers’ refers to a combination of the homoligomers or homopolymers that are the reaction product of the monomers.

A biodegradable polymer herein is preferred to have a weight average molecular weight (M_{w}) of 5,000-100,000, more preferably 10,000-20,000. The yield of nanoparticle production may be decreased and the stability of polysaccharide may be lowered due to the difficulty in molecular formation if the M_{w} is outside the aforementioned range.

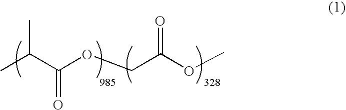

As used herein, the term “a biocompatible polymer” refers to a polymer having the tissue compatibility and the blood compatibility so that it causes neither the tissue necrosis nor the blood coagulation upon contact with tissue or blood. As used herein, the term “a biocompatible polymer emulsifier” means a biocompatible polymer that is capable of emulsifying two or more separated phases.

Examples of the biocompatible polymer emulsifier herein include, without limitation, poloxamer, poloxamine, poly(vinyl alcohol), poly(ethylene glycol) ether of alkyl alcohol and their combination. Among these polymers, poloxamer is preferred.

Examples of the biocompatible polymer emulsifier include without limitation a poloxamer, a poloxamine, a poly(vinyl alcohol), a poly(ethylene glycol) ether of alkyl alcohol and a combination thereof. Among these polymers, poly(lactide-co-glycolide) approved by FDA as non-cytotoxic is preferred.

A biocompatible polymer emulsifier herein is preferred to have a weight average molecular weight of 5,000-100,000, more preferably 10,000-20,000, and a hydrophilic portion of 60-80%. If the M_{w} is outside the aforementioned range, the yield of nanoparticle production may be decreased and the fixation of functional polysaccharide may also become difficult due to the instability of dispersion of the nanoparticles.

As used herein, the term “a polysaccharide-functionalized nanoparticle” or “a functional nanoparticle” or the like means a nanoparticle to which the functionality is endowed so that the nanoparticle may form a physical binding with a protein through a polysaccharide as shown in Preparatory Examples herein.

Meanwhile, the term “a biocompatible polymer”, which is used in “a hydrogel matrix composed of a biocompatible polymer”, “a biocompatible polymer hydrogel matrix”, “a biocompatible polymer for manufacturing a hydrogel matrix” and the like herein, is not limited to the aforementioned examples of the biocompatible polymer, and includes any kind of synthetic or natural biocompatible polymer that has been used for manufacturing a hydrogel matrix.

Examples of the biocompatible polymer for manufacturing a hydrogel matrix include without limitation synthetic polymer such as poly(ethylene glycol), poloxamer, poly(organophosphazene) and oligo(poly(ethylene glycol)fumarate); and natural polymer such as collagen, gelatin, fibrin, hyaruronic acid and alginate; and a combination thereof.

In particular, although the following Examples herein employs as a biocompatible polymer for manufacturing a hydrogel matrix only fibrin, which has cell-binding sites and glycosaminoglycanin-binding sites in a polymer, facilitates the manufacture of hydrogel and is widely clinically used as a tissue glue and a topical hemostatic agent, the present invention is not limited to fibrin nor to the aforementioned examples of the biocompatible polymer for manufacturing a hydrogel matrix in any way insofar as the polymer is biocompatible and appropriate for accomplishing the effects of the present invention, i.e. sustained-release effect and release-controlling effect.

In the present invention, a hydrogel matrix composed of a biocompatible polymer initially fills the implanted sites and also acts as a matrix, into which bone formation can occur. Moreover, a protein or a protein drug loaded in the composite may be stabilized by the formation of a specific binding with polysaccharide in nanoparticles, and may be further stabilized within the hydrogel matrix.

As used herein, the expression of “physically bound” or the like refers to any kind of non-chemical binding induced by means of other than chemical binding caused by a chemical reaction. Therefore, the physical bindings herein include without limitation a physical fixation such as an adsorption, a cohesion, an entanglement, an entrapment; and/or an electrical interaction such as a hydrogen bonding and a van der Waals interaction.

A composite, a drug delivery system for sustained release and a bone defect replacement matrix for sustained release herein are biocompatible as long as each ingredient thereof is biocompatible because the polysaccharide is physically bound to a core and/or a hydrogel layer without causing any change in structure or property due to the chemical reaction. In this regard, a composite, a drug delivery system for sustained release and a bone defect replacement matrix for sustained release according to the present invention are advantageous in terms of biocompatibility.

As used herein, the term of “a specific binding”, “a specific interaction” or the like refers to a specific binding between a protein (or a protein drug) and a polysaccharide based on their complementary structure like the receptor-ligand and the antigen-antibody interactions. The specific binding may be a covalent bond or a non-covalent bond, and particularly includes the polysaccharide-protein interaction that inhibits the hydrolysis and maintains a three-dimensional structure of the protein, thus stabilizing the protein and enhancing its biological activity.

Further, it is obvious in view of the objects of the present invention that a specific binding herein should have a sufficient binding strength for maintaining the composite herein in a relatively stable state under in vivo condition, while enabling the composite to be separated for the sustained-release effect. The aforementioned degree of the binding strength may be definitely understood by one skilled in the art with reference to related arts including, for example, “Eur. J. Biochem. 1996; 237:295-302” or “Biochem Soc Trans. 2006; 34:458-6”. The interaction between protein drug and polysaccharide decreases the initial burst of protein drug and enhances the sustained-release effect.

As used herein, the term “a polysaccharide” includes any kind of polysaccharide that may form a specific binding with various peptides or proteins such as a growth factor, a chemokine, an extracellular matrix protein and antithrombin III, thus enabling to inhibit the hydrolysis, maintain a three-dimensional structure of the protein, stabilize the protein and enhance its biological activity. The polysaccharide is preferred to have a weight average molecular weight of 3,000-100,000, more preferably 8,000-15,000 for a sufficient strength of a physical binding between a core and a hydrogel layer as well as the stability of the nanoparticles herein.

Examples of the polysaccharide include without limitation heparin of Formula (2), alginate, hyaruronic acid, chitosan and a combination thereof. Among these polysaccharides, heparin, an anionic polysaccharide approved by FDA as non-cytotoxic, is preferred.

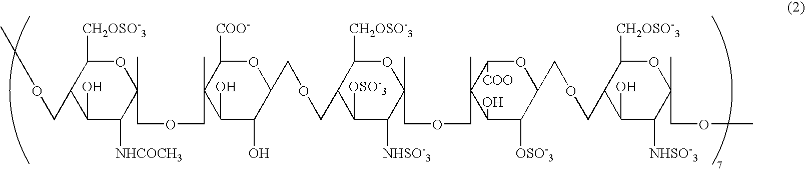

As used herein, the term “a protein drug” refers to any kind of protein or polypeptide that is capable of forming a specific binding with a polysaccharide. Examples of the protein drug include without limitation a growth factor such as BMP, VEGF, FGF, PDGF; a chemokine; an extracellular matrix protein, an antithrombin III and a combination thereof. According to a preferable embodiment, a protein drug herein may be at least one BMP selected among BMP-2, BMP-4, BMP-6, BMP-7, BMP-8 and BMP-9, which shows an osteoinductive activity.

According to an embodiment of the present invention, 2-100 μg of polysaccharide is preferred to be contained in 1 mg of nanoparticles. If the amount of polysaccharide is lower than 2 μg, it may be difficult to obtain monodisperse nanoparticles. If the amount is higher than 100 μg, the sustained release effect may decrease. Considering the release tendency and the local effective amount of drug, nanoparticles herein preferably may contain 0.01-5 μg of the growth factor relative to 1 mg of the nanoparticles.

According to another embodiment of the present invention, nanoparticles herein are preferred to have a diameter of 400 nm or less as in that the sterilization of the final product may be preformed conveniently by using a sterile filter. The surface charge of nanoparticles herein may be determined considering the target protein, and is preferred to be less than −40 mV or higher than +20 mV for the effective loading of protein into a hydrogel layer and/or a core. It is preferred that the polydispersity is less than 0.1 for the stable monodispersity distribution. Further, according to an embodiment of the present invention, a sustained release system of a growth factor shows 200-20,000 Pa of an elastic modulus (G′), which is measured after hydrogel is formed completely, for the normal survival, proliferation and differentiation of cells in composite. The elastic modules may be controlled by changing the concentration of aqueous solution of biocompatible polymer and the amount of crosslinking factors as described in the present invention.

The present invention also relates to the processes for preparing a nanoparticle-protein-hydrogel composite, a drug delivery system for sustained release and a bone defect replacement matrix for sustained release according to the present invention.

According to one aspect of the present invention, there is provided a process for preparing a nanoparticle-protein-hydrogel composite, the process comprising: (a) preparing a polysaccharide-functionalized nanoparticle, which comprises (1) dissolving a biodegradable polymer in an organic solvent which is non-cytotoxic at a low concentration, whereby preparing an organic solution, (2) dissolving a polysaccharide and a biocompatible polymer emulsifier in water, whereby preparing an aqueous solution, and (3) dispersing the organic solution in the aqueous solution; (b) loading a protein in the polysaccharide-functionalized nanoparticle, whereby preparing a polysaccharide-functionalized nanoparticle loaded with a protein; (c) dispersing the polysaccharide-functionalized nanoparticle loaded with a protein in an aqueous solution of a biocompatible polymer for manufacturing a hydrogel matrix; and (d) providing the suspension solution with at least one crosslinking means selected from the group consisting of a crosslinking agent, a crosslinking activator, a physical crosslinking factor, whereby crosslinking the biocompatible polymer for manufacturing a hydrogel matrix.

According to another aspect of the present invention, there is provided a process for preparing a drug delivery system for sustained release, where an effective amount of at least of protein drug selected among a growth factor, a chemokine, an extracellular matrix protein and antithrombin III is loaded as a protein in the step (b) of the aforementioned process for preparing a nanoparticle-protein-hydrogel composite. Especially, the protein drug may be a growth factor related to the bone formation, which is selected among BMP, TGF-beta, VEGF, FGF and PDGF.

According to still another aspect of the present invention, there is provided a process for preparing a bone defect replacement matrix for sustained release, which comprises the step of preparing a sustained release system of a growth factor according to the present invention.

According to a preferable embodiment herein, there is provided a process for preparing a nanoparticle-protein-hydrogel composite, the process comprising: (a) preparing a polysaccharide-functionalized nanoparticle, which comprises (1) dissolving at least one biodegradable polymer selected among poly(D,L-lactide-co-glycolide), poly(lactic acid), poly(glycolic acid), poly(ε-caprolactone), poly(δ-valerolactone), poly(β-hydrobutyrate) and poly(β-hydroxyvalerate) in an organic solvent which is non-cytotoxic at a low concentration, whereby preparing an organic solution, (2) dissolving (i) at least one polysaccharide selected among heparin, alginate, hyaruronic acid and chitosan and (ii) at least one biocompatible polymer emulsifier selected among poloxamer, poloxamine, poly(vinyl alcohol) and poly(ethylene glycol) ether of alkyl alcohol in water, whereby preparing an aqueous solution, and (3) dispersing the organic solution in the aqueous solution; (b) loading an effective amount of at least one growth factor selected among BMP, TGF-beta, VEGF, FGF and PDGF in the polysaccharide-functionalized nanoparticle, whereby preparing a polysaccharide-functionalized nanoparticle loaded with a protein; (c) dispersing the polysaccharide-functionalized nanoparticle loaded with a growth factor in an aqueous solution of at least one biocompatible polymer for manufacturing a hydrogel matrix selected among poly(ethylene glycol), poloxamer, poly(organophosphazene), oligo(poly(ethylene glycol)fumarate), collagen, gelatin, fibrin, hyaruronic acid and alginate; and (d) providing the suspension solution with at least one crosslinking means among a crosslinking agent such as glutaraldehyde, diepoxide and carbodiimide; a crosslinking activator such as thrombin and factor XIII; a physical crosslinking factor such as temperature, pH and specific interaction, whereby crosslinking the biocompatible polymer for manufacturing a hydrogel matrix.

With respect to the step (a), the concentration of the organic solution in the step (1) is preferred to be 0.5-2.0% (w/v) to minimize the loss due to the coagulation of biodegradable polymer when preparing nanoparticles. As considering the thickness of the hydrogel layer and the appropriate viscosity of the aqueous solution for effective particle formation, the aqueous solution in the step (2) is preferred to be so prepared that the concentration of the biocompatible polymer emulsifier may be 0.01-5% (w/v).

Organic solution containing biocompatible polymer emulsifier is dispersed in aqueous solution containing polysaccharide, and forms polysaccharide-functionalized nanoparticles. The polysaccharide is preferred to be added in an amount of 10 wt % or less relative to the weight of the biocompatible polymer emulsifier as considering polydispersity and production yield of the nanoparticles.

As considering the cytotoxicity of the organic solvent remaining in nanoparticles, the mixing ratio of the organic solution and the aqueous solution in the step (3) is preferred to be so adjusted without limitation that the volume of the organic solution is less than 10% relative to the volume of the aqueous solution.

Further, the step (b) is preferred to comprise the steps of (b′) redispersing the polysaccharide-functionalized nanoparticle in a dispersing solvent, whereby preparing a resuspension solution; and (b″) adding a solution of the growth factor in the resuspension solution.

Preferably, the concentration of the resuspension solution is preferred to be higher than 25% (w/v) as considering volume and strength of the final implant. The solution of the growth factor in the step (b″) is preferred to be prepared by using at least one solvent selected among PBS (phosphate buffered saline), PB (phosphate buffer), Tris and Hepes buffers considering the structural stability of protein. The concentration is preferred to be 0.01-0.5% (w/v), considering the volume and strength of the final implant.

Further, in the step (c) above, the hydrogel matrix may be prepared (i) by dispersing the prepared polysaccharide-functionalized nanoparticle loaded with a grow factorin an aqueous solution of at least one biocompatible polymer for manufacturing a hydrogel matrix selected among poly(ethylene glycol), poloxamer, poly(organophosphazene), oligo(poly(ethylene glycol)fumarate), collagen, gelatin, fibrin, hyaruronic acid and alginate as described in the step (c). Alternatively, the hydrogel matrix may also be prepared (ii) by dispersing the nanoparticles in an aqueous solution of monomers or oligomers of the aforementioned biocompatible polymer, and polymerizing and/or crosslinking the monomers or oligomers.

Preferably, the amount of a crosslinking agent, a crosslinking activator, temperature, pH, a specific interaction, which is to be supplied for crosslinking biocompatible polymer for manufacturing a hydrogel matrix in the step (d), may be determined so that an elastic modulus (G′), which is measured after hydrogel is formed completely, is within 200-20,000 Pa for the normal survival, proliferation and differentiation of cells in composite.

According to an embodiment, a process for preparing a bone defect replacement matrix for sustained release may comprise the step of (e) molding the sustained release system of a growth factor so that the molded system may fit to a defect of a bone or an alveolar bone formed due to at least one selected from the group consisting of osteoporosis, fracture of a bone, fracture dislocation, non-union, delayed union, bone defect, alveolar bone defect.

Before and/or after and/or at the same time with performing the step (e), it is preferred to add at least one selected among (i) autogenous bone without cells, allogeneic bone and xenogeneic bone; (ii) HAP, tricalcium phosphate, calcium aluminate, β-TCP, CPC, calcium sulfate and bioglass®; and (iii) a cell-binding protein and a degradable peptide linker.

As used herein, “an organic solvent which is non-cytotoxic at a low concentration” refers to an organic solvent that has been reported as non-cytotoxic at such a low concentration that the organic solvent may remain within nanoparticles. Examples of the organic solvent include without limitation dimethylsulfoxide (‘DMSO’ hereinafter) or tetraglycol, both of which are reported as non-cytotoxic at a concentration of less than 10% (w/w).

Any “water” may be used in the present invention only if it is biocompatible and non-cytotoxic, and is not limited to distilled water. Further, any conventional method may be used herein to add and disperse/emulsify organic solvent in an aqueous solvent.

As used herein, the term “an effective amount” is such a minimal amount that systems in various embodiments herein may exert the therapeutic or prophylactic efficacy herein. Particularly, the therapeutic or prophylactic efficacy herein includes the treatment or prophylaxis of bone defects as well as the promotion of bone formation in bone defect sites. Causes of the bone defects include without limitation osteoporosis, limb or spine fractures, fracture dislocation, non-union, delayed union, osteomyelitis, tumor ablation in orthopedics, alveolar defects in dentistry bone loss, alveolar bone loss and a combination thereof.

One skilled in the art may easily determine the effective amount by considering various factors such as kind of disease, severity of disease, kind or amount of ingredients, type of formulation, route or time of administration, period of treatment, age, body weight, physical conditions, sex of a patient, food, release rate and other drug to be used together.

Furthermore, the present invention also relates to a method controlling the release rate of a protein drug in the aforementioned drug delivery system for sustained release according to the present invention.

Especially, the release rate of a protein drug is controlled: (A) by changing the content of the polysaccharide in a unit mass of the nanoparticle by means of (i) changing the concentration of the polysaccharide in the aqueous solution in the step (a) (2) and/or (ii) changing the mixing ratio of the organic solution and the aqueous solution in the step (a)(3); and/or (B) by changing the content of the nanoparticle in a unit mass of the composite in the aqueous solution of a biocompatible polymer for manufacturing a hydrogel matrix in the step (c) by means of changing the concentration ratio between the polysaccharide-functionalized nanoparticle and the biocompatible polymer for manufacturing a hydrogel matrix.

Further, it is obvious that one skilled in the art may easily control the content of the polysaccharide in a unit mass of the nanoparticle by changing the concentration of the polysaccharide in the aqueous solution and/or by changing the mixing ratio of the organic solution and the aqueous solution based on the description herein, although its specific procedure is not described in Examples herein. Likewise, one skilled in the art may also easily control the content of the nanoparticle in a unit mass of the composite in the aqueous solution of a biocompatible polymer for manufacturing a hydrogel matrix by changing the concentration ratio between the polysaccharide-functionalized nanoparticle and the biocompatible polymer for manufacturing a hydrogel matrix based on the description herein.

### EXAMPLES

The present invention is described more specifically by the following Examples. Examples herein are meant only to illustrate the present invention, but in no way to limit the claimed invention.

The paper of “Biomaterials 27 (2006) 2621-2626” is incorporated by reference herein in their entirety for better understanding of the gist of the present invention, especially of the experimental process herein.

A. Step 1: Nanoparticles

### 1. Comparative Preparatory Example

### Preparation of Non-Functionalized Nanoparticles with Hydrophilic Hydrogel Layer (PLGA NP)

40 mg of PLGA was completely dissolved in 2 mL of dimethylsulfoxide, and this solution was slowly added in 30 mL of 5% aqueous solution of poloxamer, thus providing non-functionalized nanoparticles. Remaining poloxamer and dimethylsulfoxide were removed by performing high-speed centrifugation, followed by separation of supernatant liquid. Thus obtained nanoparticles were resuspended in distilled water or PBS (phosphate buffered saline) solution (pH 7.4).

### 2. Preparatory Examples 1-5

### Preparation of Heparin-Functionalized Nanoparticles (HEP-PLGA NP)

40 mg of PLGA was completely dissolved in 2 mL of dimethylsulfoxide, and 2 mL of this solution was slowly added in each of 5% aqueous poloxamer solution (30 mL), which contains 10, 30, 60, 120 and 240 mg of heparin, respectively, thus providing heparin-functionalized nanoparticles. Remaining excess heparin, poloxamer and dimethylsulfoxide were removed by performing high-speed centrifugation, followed by separation of supernatant liquid. Thus obtained nanoparticles were resuspended in distilled water or PBS solution.

### 3. Experimental Preparatory Example

### Observation of Size, Surface Charge, Contents and Polydispersity of Heparin-Functionalized Nanoparticles (HEP-PLGA NP)

The size and the surface charge of the prepared nanoparticles were measured according to the dynamic light scattering method and the electrophoretic light scattering method, respectively, by using ELS-8000 (Otsuka Electronics Co., Japan).

The size increased from 123.1±2.0 nm to 188.1±3.9 and the surface charge varied from −26.0±1.1 mV to −44.4±1.2 mV with the increase of heparin amount in the aqueous solution of poloxamer. As the heparin carries a strong negative charge, the relatively higher negative value in surface charge means that a higher amount of heparin exists on the surface of the nanoparticles.

Dry weight of the nanoparticles was calculated after freeze-drying the nanoparticles. The partial amount of the heparin in the hydrogel layer and the total amount of the heparin in the nanoparticles were calculated through an anti-factor Xa analysis (C. Chauvierre et al., Biomaterials, 25 (2004) 3081-3086) by using particle-state nanoparticles and the nanoparticle solution, respectively. The ratio of PLGA to poloxamer in nanoparticles was finally obtained by performing ¹H NMR analysis to determine the mass ratio of each ingredient. The results are presented in Table 1.

As shown in Table 1, most of physically-bound heparin was ascertained to exist in a surface layer. Further, high-speed centrifugation removed non-bound heparin including heparin that was just dispersed in hydrogel layer, which shows that the heparin exiting in a surface layer is physically bound to the hydrogel surface layer comprising poloxamer and/or a core. It is assured that poloxamer is stabilized by a hydrophobic interaction with PLGA and that heparin is fixed to the surface layer via hydrophilic interaction between the carboxylic groups in heparin and poly(ethylene glycol) in poloxamer.

Table 1 provides contents of each ingredient in nanoparticles prepared in Comparative Preparatory Example 1 and Preparatory Examples 2 and 4.

TABLE 1
Content of Each Ingredient in Nanoparticles

| | | | | Total amount |
|---|---|---|---|---|
| Heparin in | | | Heparin in | of heparin in |
| aqueous solution | PLGA | Poloxamer | surface layer | nanoparticles |
| 0 mg | 36.8 ± 1.6 mg | 13.8 ± 0.6 mg | 0.0 mg | 0.0 mg |
| | (72.7%) | (27.3%) | (0.0%) | (0.0%) |
| 30 mg | 34.7 ± 0.9 mg | 13.3 ± 0.4 mg | 0.94 ± 0.04 mg | 1.20 ± 0.04 mg |
| | (70.6%) | (27.0%) | (1.9%) | (2.4%) |
| 120 mg | 29.0 ± 1.8 mg | 12.3 ± 0.8 mg | 1.71 ± 0.09 mg | 2.01 ± 0.10 mg |
| | (66.9%) | (28.4%) | (4.0%) | (4.7%) |

Table 1 shows that the amount of heparin in hydrogel surface layer increases with the increase of the amount of heparin in an aqueous poloxamer solution. However, the aqueous solution is preferred to contain heparin in the concentration of 120 mg/2 mL or less as considering polydispersity and production yield of nanoparticles. Especially, nanoparticles were so prepared as to contain 0 wt %, 2.4 wt % and 4.7 wt % of heparin when the amount of heparin in the poloxamer aqueous solution is 0, 30 120 mg, respectively.

B. Step 2: Nanoparticles Loaded with Protein

### 1. Comparative Preparatory Example

### Preparation of Non-Functionalized Nanoparticles Loaded with Lysozyme (Lysozyme-Loaded PLGA NP)

The non-functionalized nanoparticles prepared in Comparative Preparatory Example of Step 1 above were collected by high-speed centrifugation, resuspended in PBS solution and loaded with 1 mg of lysozyme as a model protein.

### 2. Preparatory Examples 1-2

### Preparation of Heparin-Functionalized Nanoparticles Loaded with Lysozyme (Lysozyme-Loaded HEP-PLGA NP)

The nanoparticles prepared in Preparatory Examples 2 and 4 of Step 1 were collected by high-speed centrifugation, resuspended in PBS solution and loaded with 1 mg of lysozyme as a model protein, respectively.

### 3. Preparatory Examples 3-4

### Preparation of Heparin-Functionalized Nanoparticles Loaded with VEGF (VEGF-Loaded HEP-PLGA NP)

Following the procedure described in Preparatory Examples 1-2, the nanoparticles prepared in Preparatory Example 4 of Step 1 were loaded with VEGF. One group of nanoparticles was loaded with 15.6 ng of VEGF and another group was loaded with 156 ng of VEGF relative to 1 mg of the nanoparticles.

### 4. Preparatory Example 5

### Preparation of Heparin-Functionalized Nanoparticles Loaded with BMP (BMP-Loaded HEP-PLGA NP)

The nanoparticles prepared in Preparatory Example 4 of Step 1 were used. Remaining excess heparin, poloxamer and dimethylsulfoxide were removed by performing high-speed centrifugation, followed by separation of supernatant liquid. The obtained nanoparticles were resuspended in 40 μL of PBS solution and mixed with 156 ng of BMP relative to 1 mg of nanoparticles, followed by incubation at 4° C. overnight with gentle rotation, thus preparing nanoparticles loaded with BMP-2 (Peprotech, cat# 120-02).

### 5. Experimental Preparatory Example 2

### In Vitro Observation of Sustained Release and Stabilizing Effect of Lysozyme (Lysozyme-Loaded PLGA NP & Lysozyme-Loaded HEP-PLGA NP)

In vitro release behavior was observed to ascertain the sustained release of lysozyme and the stability of protein drug by using the systems (i.e. the nanoparticles loaded with drug) prepared in Comparative Preparatory Example and Preparatory Examples 1-2 of Step 2.

After suspension solution of nanoparticles loaded with lysozyme was placed in a dialysis tube (MWCO 500 k), the released lysozyme was collected by using a large amount of PBS solution under the infinite dilution condition. The amount of the collected lysozyme was quantified according to the Micro BCA protein quantification. PBS used for collecting lysozyme was replaced with new one every day, and the sample was stored at 4° C. until the protein quantification was performed.

The nanoparticles with no heparin released about two thirds amount of the loaded drug within 3 days, while the release of the nanoparticles with 4.7 wt % of heparin lasted for up to 19 days without an initial burst. It was also ascertained that the increase in heparin amount in turn enhances the effect of the sustained release.

Biological activity of the released lysozyme was observed, and it was ascertained that the loaded protein drug was stabilized by the heparin fixed to the hydrogel surface layer.

### 6. Experimental Preparatory Example 3

### In Vitro Observation of Sustained Release and Stabilizing Effect of BMP (BMP-Loaded HEP-PLGA NP)

In vitro sustained release of BMP was observed by using the system (i.e. the nanoparticles loaded with drug) prepared in Preparatory Example 5. After suspension solution of nanoparticles loaded with BMP was placed in a dialysis tube (MWCO 500 k), the released BMP was collected by using a large amount of PBS solution under the infinite dilution condition. The amount of the collected BMP was quantified by using ELISA (enzyme-linked immunosorbent assay). PBS used for collecting BMP was replaced with new one every day, and the sample was stored at −30° C. until the protein quantification was performed.

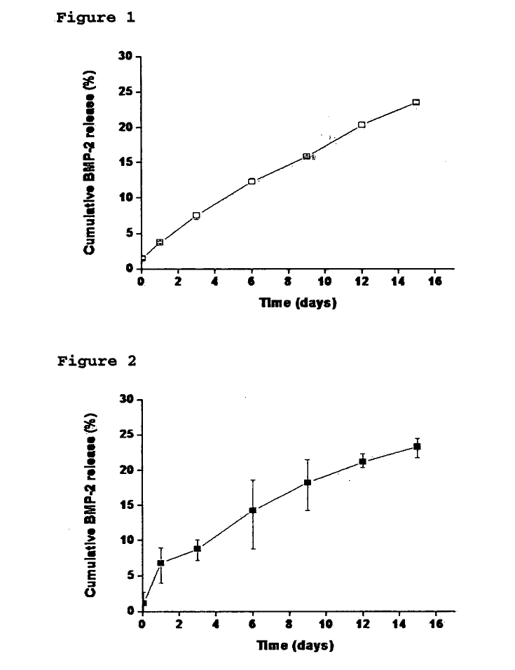

The nanoparticles, which contain 4.7 wt % of heparin and are loaded with 156 ng of BMP relative to 1 mg of the nanoparticles, released BMP up to 24 wt % of the initial loaded amount for 15 days without an initial burst (FIG. 1). This result ascertains the applicability of heparin-functionalized nanoparticle according to the present invention in the use of delivering BMP for sustained release.

C. Step 3: Heparin-Functionalized Composite Loaded with BMP (Drug Delivery System)

### 1. Example 1

### Preparation of Heparin-Functionalized Composite Loaded with BMP (Drug Delivery System; BMP-Loaded HEP-NP-Hydrogel Composite)

The nanoparticles collected in Preparatory Example 4 of Step 1 were resuspended and loaded with BMP, to prepare a final suspension solution. 26 μL of the final suspension solution was uniformly mixed with 70 μL of aqueous fibrinogen solution (7-12%, w/v), and further mixed with 70 μL of a solution containing thrombin as a crosslinking activator, thus providing nanoparticle-hydrogel composite.

The aqueous fibrinogen solution contained fibrinogen (71.5-126.5 mg), factor XIII (44-88 U) and aprotinin (1,100 KIU) as main ingredients, and was prepared by dissolving freeze-dried fibrinogen in an aqueous solution containing the other ingredients. The aqueous solution was adjusted so as to finally contain 65-115 mg of fibrinogen, 40-80 U of factor and 1,000 KIU of aprotinin per mL of solution.

The thrombin solution was prepared by dissolving freeze-dried thrombin in an aqueous solution containing the other ingredients. The thrombin solution was adjusted so as to finally contain 400-600 IU of thrombin in 1.2 mL of 0.6% (w/v) potassium chloride solution.

After the two solutions were mixed, the mixture was incubated at room temperature for 30 minutes.

### 2. Experimental Example 1

### In Vitro Observation of Sustained Release of BMP (BMP-Loaded HEP-NP-Hydrogel Composite)

BMP release behavior was observed under the following in vitro conditions by using the composite (drug delivery system) prepared in Example 1 of Step 3.

After the BMP-loaded heparin-functionalized composite was placed in a bottle containing release buffer, the released BMP was collected by using a large amount of PBS solution under the infinite dilution condition. The amount of the collected BMP was quantified by using ELISA (enzyme-linked immunosorbent assay). PBS used for collecting lysozyme was replaced with new one every day, and the sample was stored at −30° C. until the protein quantification was performed.

The composite, which contains 4.7 wt % of heparin and is loaded with 156 ng of BMP relative to 1 mg of the composite, released BMP up to 23 wt % of the initial loaded amount for 15 days without an initial burst (FIG. 2). This result ascertains the applicability of heparin-functionalized composite according to the present invention in the use of delivering BMP for sustained release.

D. Step 4: Heparin-Functionalized Bone Defect Replacement Matrix Loaded with BMP

### 1. Example 1

### Preparation of Bone Defect Replacement Matrix comprising heparin-functionalized composite loaded with Protein (BMP-Loaded HEP-NP-Hydrogel Bone Defect Replacement Matrix)

The composite prepared in Example 1 of Step 3 was molded in a disc-type mold as described below. In particular, the final disc-type system was so prepared as to be applied to a rat calvarial bone defect model (8 mm in diameter and 2.1 mm in height).

The fibrinogen solution including the BMP loaded nanoparticles was placed into a disc-type mold with a flat bottom and an inside diameter of 8 mm. Fibrin gel formation was initiated by adding 70 μL thrombin solution, in which 500 IU of thrombin from human plasma was dissolved with 1.2 mL of 0.6% (w/v) calcium chloride solution, and then incubated for 30 min at room temperature in a humid atmosphere.

The gellation of the nanoparticle-fibrin gel complex was monitored using a rheometer (Gemini, Malvern Instruments, UK). A sample holder having parallel plate geometry (gap: 0.3 mm, diameter: 15 mm) with a roughened surface to prevent slippage and a solvent trap to prevent drying during measurements was used. An oscillatory time sweep at 1 rad/s frequency with 0.1% shear strain was used in the linear viscoelastic range.

### 2. Experimental Example and Comparative Experimental Example

### Evaluation of In-Vivo Bone Regeneration of Bone Defect Replacement Matrix Loaded with BMP for Sustained Release (Exp. Ex.) and Hydrogel Matrix (Comp. Exp. Ex.)

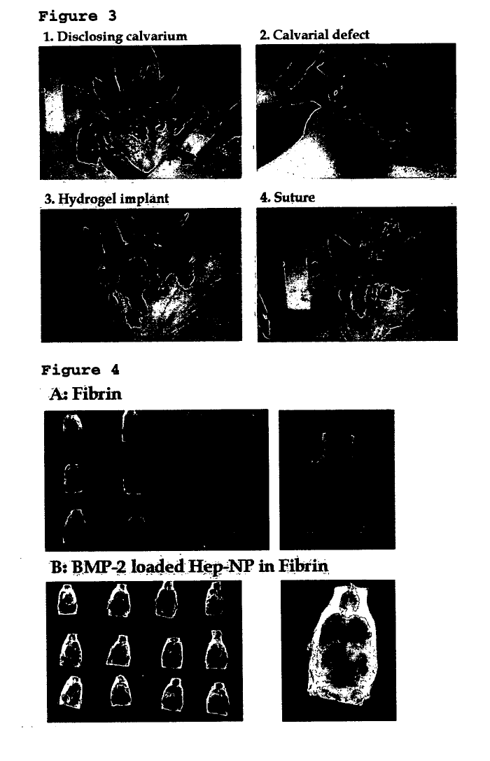

As the Experimental Example, in-vivo bone regeneration of bone defect replacement matrix loaded with BMP for sustained release was observed by using a rat calvarial bone defect model as described below. The bone defect replacement matrix prepared in Example 1 of Step 4 was implanted into bone defect sites of 12 rats (FIG. 3). As the Comparative Experimental Example, bone defect sites of 12 rats were filled only with hydrogel matrix instead of bone defect replacement matrices under the same conditions. However, as considering that bone formation may be delayed by bleeding during the step of inducing the calvarial bone defect, the bleeding problem was attempted to be minimized by performing preliminary experiments.

The animals were sacrificed after 4 weeks of surgery and subject to radiological evaluation by using a soft x-ray (30 KVP, 1.5 mA, distance: 52 cm, exposure time: 90 seconds). Bone formation was observed in all the twelve rats used in Experimental Example 2. Some specimen in Experimental Example showed remarkably improved bone formation as compared to Comparative Experimental Example although calvarial bone defect was not perfectly regenerated in the specimen (FIG. 4).

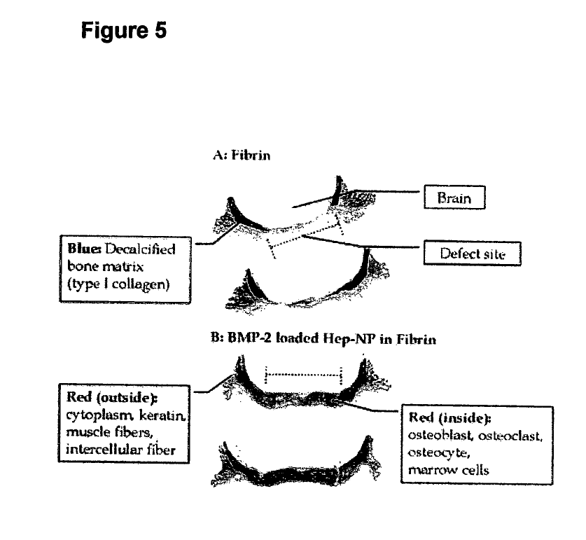

Further, for the histological analysis, bone formation was observed by staining decalcified sections with Masson's trichrome stain (MT). The complete formation of bone is ascertained in a photograph of Experimental Example 2, including collagen, osteocytes isolated inside collagen, marrow cells, and osteoblasts and osteoclasts existing in the marginal region between collagen and marrow cells. In contrast, Comparative Experimental Example is remarkably inferior to Experimental Example 2 although Comparative Experimental Example shows partial bone regeneration at the peripheral edge of the bone defect sites (FIG. 5). This result ascertains that bone defect replacement matrix according to the present invention is superior in an osteoinductive activity.

As described above, a composite, a drug delivery system or a bone defect replacement matrix comprising the composite for sustained release herein is advantageous in that: (i) it is more easily prepared than the conventional osteoinductive system, (ii) it may ameliorate the financial burden of patients, (iii) it improves a bone regeneration remarkably, (iv) nanoparticle-hydrogel composite system may be easily applied for the delivery of other important growth factors, thus enabling the recovery of other tissues. Therefore, a bone defect replacement matrix for sustained release using BMP delivery system herein may be widely applied to orthopedics, plastic surgery and dentistry, and is expected to remarkably increase the market size besides the established market.

1. A nanoparticle-protein-hydrogel composite comprising:
(a) a polysaccharide-functionalized nanoparticle comprising:
(1) a core composed of a biodegradable polymer,
(2) a hydrogel surface layer composed of a biocompatible polymer emulsifier, and
(3) a polysaccharide physically bound to the core and/or the hydrogel layer;
(b) a protein forming a specific binding with the polysaccharide; and
(c) a hydrogel matrix composed of a biocompatible polymer as a matrix for the nanoparticle.

2. A drug delivery system for sustained release comprising:
(a) a polysaccharide-functionalized nanoparticle comprising:
(1) a core composed of a biodegradable polymer,
(2) a hydrogel surface layer composed of a biocompatible polymer emulsifier, and
(3) a polysaccharide physically bound to the core and/or the hydrogel layer;
(b) an effective amount of a protein selected from the group consisting of a growth factor, a chemokine, an extracellular matrix protein, an antithrombin III and a combination thereof, which forms a specific binding with the polysaccharide; and
(c) a hydrogel matrix composed of a biocompatible polymer as a matrix for the nanoparticle.

3. A sustained release system of a growth factor comprising:
(a) a polysaccharide-functionalized nanoparticle comprising:
(1) a core composed of a biodegradable polymer selected from the group consisting of the group consisting of a poly(D,L-lactide-co-glycolide), a poly(lactic acid), a poly(glycolic acid), a poly(ε-caprolactone), a poly(δ-valerolactone), a poly(β-hydrobutyrate), a poly(β-hydroxyvalerate) and a combination thereof,
(2) a hydrogel surface layer composed of a biocompatible polymer emulsifier selected from the group consisting of a poloxamer, a poloxamine, a poly(vinyl alcohol), a poly(ethylene glycol) ether of an alkyl alcohol and a combination thereof, and
(3) a polysaccharide physically selected from the group consisting of a heparin, an alginate, a hyaruronic acid, a chitosan and a combination thereof, which is bound to the core and/or the hydrogel layer;
(b) an effective amount of a growth factor selected from the group consisting of BMP, TGF-beta, VEGF, FGF, PDGF and a combination thereof, which forms a specific binding with the polysaccharide; and
(c) a hydrogel matrix composed of a biocompatible polymer selected from the group consisting of a poly(ethylene glycol), a poloxamer, a poly(organophosphazene), an oligo(poly(ethylene glycol)fumarate), a collagen, a gelatin, a fibrin, a hyaruronic acid, an alginate and a combination thereof as a matrix for the nanoparticle.

4. The sustained release system of claim 3, wherein the biodegradable polymer is an poly(D,L-lactide-co-glycolide); the biocompatible polymer emulsifier is a poloxamer; the polysaccharide is a heparin; the growth factor is selected from the group consisting of BMP-2, BMP-4, BMP-6, BMP-7, BMP-8, BMP-9, TGF-beta, VEGF, FGF, PDGF and a combination thereof; and the hydrogel matrix is a fibrin.

5. The sustained release system of claim 4, wherein the biodegradable polymer and the biocompatible polymer emulsifier have a weight average molecular weight of 5,000-100,000, respectively; 2-100 μg of the polysaccharide and 0.01-5 μg of the growth factor are contained in 1 mg of the nanoparticle; the nanoparticle has a diameter of 400 nm or lower, a surface charge of higher than +20 mV or lower than −40 mV and a polydispersity of lower than 0.1; and the sustained release system has an elastic modules of 200-20,000 Pa.

6. A bone defect replacement matrix for sustained release comprising the sustained release system of a growth according to claim 3.

7. The bone defect replacement matrix of claim 6, which is used for the treatment or prophylaxis of osteoporosis, fracture of a bone, fracture dislocation, non-union, delayed union, bone defect, alveolar bone defect and a combination thereof.

8. The bone defect replacement matrix of claim 7, which further comprises at least one selected from the group consisting of (i) autogenous bone without cells, allogeneic bone and xenogeneic bone; (ii) HAP, tricalcium phosphate, calcium aluminate, β-TCP, CPC, calcium sulfate and bioglass®; and (iii) a cell-binding protein and a degradable peptide linker.

9. A process for preparing a drug delivery system for sustained release, the process comprising:
(a) preparing a polysaccharide-functionalized nanoparticle, which comprises:
(1) dissolving a biodegradable polymer in an organic solvent which is non-cytotoxic at a low concentration, whereby preparing an organic solution,
(2) dissolving a polysaccharide and a biocompatible polymer emulsifier in water, whereby preparing an aqueous solution, and
(3) dispersing the organic solution in the aqueous solution;
(b) loading a protein in the polysaccharide-functionalized nanoparticle, whereby preparing a polysaccharide-functionalized nanoparticle loaded with the protein;
(c) dispersing the polysaccharide-functionalized nanoparticle loaded with a protein in an aqueous solution of a biocompatible polymer for manufacturing a hydrogel matrix, whereby preparing a suspension solution; and
(d) providing the suspension solution with at least one crosslinking means selected from the group consisting of a crosslinking agent, a crosslinking activator, a physical crosslinking factor, whereby crosslinking the biocompatible polymer for manufacturing a hydrogel matrix.

10. A process for preparing a drug delivery system for sustained release, the process comprising:
(a) preparing a polysaccharide-functionalized nanoparticle, which comprises:
(1) dissolving a biodegradable polymer in an organic solvent which is non-cytotoxic at a low concentration, whereby preparing an organic solution,
(2) dissolving a polysaccharide and a biocompatible polymer emulsifier in water, whereby preparing an aqueous solution, and
(3) dispersing the organic solution in the aqueous solution;
(b) loading an effective amount of a protein drug selected from the group consisting of a growth factor, a chemokine, an extracellular matrix protein, an antithrombin III and a combination thereof in the polysaccharide-functionalized nanoparticle, whereby preparing a polysaccharide-functionalized nanoparticle loaded with the protein drug;
(c) dispersing the polysaccharide-functionalized nanoparticle loaded with a protein in an aqueous solution of a biocompatible polymer for manufacturing a hydrogel matrix, whereby preparing a suspension solution; and
(d) providing the suspension solution with at least one crosslinking means selected from the group consisting of a crosslinking agent, a crosslinking activator, a physical crosslinking factor, whereby crosslinking the biocompatible polymer for manufacturing a hydrogel matrix.

11. A process for preparing a sustained release system of a growth factor, the process comprising:
(a) preparing a polysaccharide-functionalized nanoparticle, which comprises
(1) dissolving at least one biodegradable polymer selected from the group consisting of a poly(D,L-lactide-co-glycolide), a poly(lactic acid), poly(glycolic acid), a poly(ε-caprolactone), a poly(δ-valerolactone), a poly(β-hydrobutyrate) and a poly(β-hydroxyvalerate) in an organic solvent which is non-cytotoxic at a low concentration, whereby preparing an organic solution,
(2) dissolving (i) at least one polysaccharide selected from the group consisting of heparin, alginate, hyaruronic acid and chitosan and (ii) at least one biocompatible polymer emulsifier selected from the group consisting of a poloxamer, a poloxamine, a poly(vinyl alcohol) and a poly(ethylene glycol) ether of alkyl alcohol in water, whereby preparing an aqueous solution, and
(3) dispersing the organic solution in the aqueous solution;
(b) loading an effective amount of a growth factor selected from the group consisting of BMP, TGF-beta, VEGF, FGF, PDGF and a combination thereof in the polysaccharide-functionalized nanoparticle, whereby preparing a polysaccharide-functionalized nanoparticle loaded with the growth factor;
(c) dispersing the polysaccharide-functionalized nanoparticle loaded with the growth factor in an aqueous solution of at least one biocompatible polymer for manufacturing a hydrogel matrix selected from the group consisting of a poly(ethylene glycol), a poloxamer, a poly(organophosphazene), an oligo(poly(ethylene glycol)fumarate), a collagen, a gelatin, a fibrin, a hyaruronic acid and an alginate, whereby preparing a suspension solution; and
(d) providing the suspension solution with at least one crosslinking means selected from the group consisting of a crosslinking agent selected from the group consisting of a glutaraldehyde, a diepoxide and a carbodiimide; a crosslinking activator selected from the group consisting of thrombin, factor XIII and a combination thereof; a physical crosslinking factor selected from the group consisting of temperature, pH and an interaction, whereby crosslinking the biocompatible polymer for manufacturing a hydrogel matrix.

12. The process of claim 11, wherein the step (b) comprises:
(b′) redispersing the polysaccharide-functionalized nanoparticle in a dispersing solvent, whereby preparing a resuspension solution; and
(b″) adding a solution of the growth factor in the resuspension solution.

13. The process of claim 12, wherein the organic solution in the step (a)(1) has the concentration of 0.5-2.0% (w/v); the aqueous solution in the step (a)(2) has the concentration of 0.01-5% (w/v) and the polysaccharide in the step (a)(2) is used in the amount of less than 10 wt % relative to the biocompatible polymer emulsifier; the organic solution in the step (a)(3) is used in the amount of less than 10 vol % relative to the aqueous solution;
the resuspension solution in the step (b′) has the concentration of higher than 25% (w/v); and
the solution of the growth factor in the step (b″) is prepared by using at least one solvent selected from the group consisting of PBS, PB, Tris and Hepes buffer, and has the concentration of 0.01-0.5% (w/v).

14. The process of claim 13, wherein the biodegradable polymer is poly(D,L-lactide-co-glycolide); the biocompatible polymer emulsifier is a poloxamer; the polysaccharide is a heparin; the growth factor is selected from the group consisting of BMP-2, BMP-4, BMP-6, BMP-7, BMP-8, BMP-9, TGF-beta, VEGF, FGF, PDGF and a combination thereof; and the hydrogel matrix is a fibrin.

15. The process of claim 14, wherein the biodegradable polymer, the biocompatible polymer emulsifier and the polysaccharide has a weight average molecular weight of 5,000-100,000, 5,000-100,000 and 3,000-100,000, respectively; 2-100 μg of the polysaccharide and 0.01-5 μg of the growth factor are contained in 1 mg of the nanoparticle;
the nanoparticle has a diameter of less than 400 nm, a surface voltage of higher than +20 mV or lower than −40 mV, and a polydispersity index of less than 0.1; and
the sustained release system of a growth factor has an elastic modules of 200-20,000 Pa.

16. A process of preparing a bone defect replacement matrix for sustained release, the process comprising:
(a) preparing a sustained release system of a growth factor according to claim 11; and
(b) molding the sustained release system of a growth factor so that the molded system may fit to a defect of a bone or an alveolar bone formed due to at least one selected from the group consisting of osteoporosis, fracture of a bone, fracture dislocation, non-union, delayed union, bone defect, alveolar bone defect.

17. The process of claim 16, wherein at least one selected from the group consisting of (i) autogenous bone without cells, allogeneic bone and xenogeneic bone;
(ii) HAP, tricalcium phosphate, calcium aluminate, β-TCP, CPC, calcium sulfate and bioglass®; and (iii) a cell-binding protein and a degradable peptide linker is added while performing the step (e).

18. A method of controlling the release rate of a protein drug, the method comprising:
(a) preparing a polysaccharide-functionalized nanoparticle, which comprises:
(1) dissolving a biodegradable polymer in an organic solvent which is non-cytotoxic at a low concentration, whereby preparing an organic solution,
(2) dissolving a polysaccharide and a biocompatible polymer emulsifier in water, whereby preparing an aqueous solution, and
(3) dispersing the organic solution in the aqueous solution;
(b) loading an effective amount of a protein drug selected from the group consisting of a growth factor, a chemokine, an extracellular matrix protein, an antithrombin III and a combination thereof in the polysaccharide-functionalized nanoparticle, whereby preparing a polysaccharide-functionalized nanoparticle loaded with the protein drug;
(c) dispersing the polysaccharide-functionalized nanoparticle loaded with a protein in an aqueous solution of a biocompatible polymer for manufacturing a hydrogel matrix, whereby preparing a suspension solution;
(d) providing the suspension solution with at least one crosslinking means selected from the group consisting of a crosslinking agent, a crosslinking activator, a physical crosslinking factor, whereby crosslinking the biocompatible polymer for manufacturing a hydrogel matrix;
wherein the release rate of a protein drug is controlled:
(A) by changing the content of the polysaccharide in a unit mass of the nanoparticle by means of (i) changing the concentration of the polysaccharide in the aqueous solution in the step (a)(2) and/or (ii) changing the mixing ratio of the organic solution and the aqueous solution in the step (a)(3); and/or
(B) by changing the content of the nanoparticle in a unit mass of the composite in the aqueous solution of a biocompatible polymer for manufacturing a hydrogel matrix in the step (c) by means of changing the concentration ratio between the polysaccharide-functionalized nanoparticle and the biocompatible polymer for manufacturing a hydrogel matrix.

19. A method of controlling the release rate of a protein drug, the method comprising:
(a) preparing a polysaccharide-functionalized nanoparticle, which comprises:
(1) dissolving at least one biodegradable polymer selected from the group consisting of a poly(D,L-lactide-co-glycolide), a poly(lactic acid), a poly(glycolic acid), a poly(ε-caprolactone), a poly(δ-valerolactone), a poly(β-hydrobutyrate) and a poly(β-hydroxyvalerate) in an organic solvent which is non-cytotoxic at a low concentration, whereby preparing an organic solution,
(2) dissolving (i) at least one polysaccharide selected from the group consisting of a heparin, an alginate, a hyaruronic acid and a chitosan and (ii) at least one biocompatible polymer emulsifier selected from the group consisting of a poloxamer, a poloxamine, a poly(vinyl alcohol) and a poly(ethylene glycol) ether of alkyl alcohol in water, whereby preparing an aqueous solution, and
(3) dispersing the organic solution in the aqueous solution;
(b) loading an effective amount of a protein drug selected from the group consisting of a growth factor selected from the group consisting of BMP, TGF-beta, VEGF, FGF, PDGF and a combination thereof in the polysaccharide-functionalized nanoparticle, whereby preparing a polysaccharide-functionalized nanoparticle loaded with the protein drug;
(c) dispersing the polysaccharide-functionalized nanoparticle loaded with a protein in an aqueous solution of a biocompatible polymer for manufacturing a hydrogel matrix selected from the group consisting of a poly(ethylene glycol), a poloxamer, a poly(organophosphazene), an oligo(poly(ethylene glycol)fumarate), a collagen, a gelatin, a fibrin, a hyaruronic acid, an alginate and a combination thereof, whereby preparing a suspension solution; and
(d) providing the suspension solution with at least one crosslinking means selected from the group consisting of a crosslinking agent selected from the group consisting of glutaraldehyde, diepoxide and carbodiimide; a crosslinking activator selected from the group consisting of thrombin, factor XIII and a combination thereof; a physical crosslinking factor selected from the group consisting of temperature, pH and an interaction, whereby crosslinking the biocompatible polymer for manufacturing a hydrogel matrix;
wherein the release rate of a protein drug is controlled:
(A) by changing the content of the polysaccharide in a unit mass of the nanoparticle by means of (i) changing the concentration of the polysaccharide in the aqueous solution in the step (a)(2) and/or (ii) changing the mixing ratio of the organic solution and the aqueous solution in the step (a)(3); and/or
(B) by changing the content of the nanoparticle in a unit mass of the composite in the aqueous solution of a biocompatible polymer for manufacturing a hydrogel matrix in the step (c) by means of changing the concentration ratio between the polysaccharide-functionalized nanoparticle and the biocompatible polymer for manufacturing a hydrogel matrix.

20. The method of claim 19, wherein the protein drug is at least one selected from the group consisting of a growth factor selected from the group consisting of BMP, VEGF, bGFG, FGF and PDGF; a chemokine; an extracellular matrix protein; and an antithrombin III.
